(19) United States
(12) Patent Application Publication
(10) Pub. No.: US 2006/0052700 A1
(43) Pub. Date: Mar. 9, 2006

(54) Pressure measurement system

(75) Inventor: Johan Svanerudh, Uppsala (SE)

Correspondence Address: FOLEY AND LARDNER LLP;SUITE 500, 3000 K STREET NW, WASHINGTON, DC, 20007, US

(73) Assignee: RADI MEDICAL SYSTEMS AB

(21) Appl. No.: 10/935,720

(22) Filed: Sep. 8, 2004

(51) Int. Cl. A61B 8/00

(52) U.S. Cl. 600/438

(57) ABSTRACT

Pressure measurement system, comprising a pressure detection device comprising a pressure sensor guidewire 5 provided with a pressure sensor connected to an electrical carrier for transmitting pressure data from the sensor to a processing means 1. The guidewire is adapted to be inserted into a vessel of a subject, and an image data capturing device for capturing image data representative of the vessel. The processing means 1 comprising a computer program product which comprises computer executable instructions for manipulating image data and pressure data to generate an output in which the pressure data is mapped onto a corresponding position on an image where that pressure data was detected to provide an integrated graphical image output on a monitoring means 2.

### FIELD OF THE INVENTION

The present invention relates to a pressure measurement system according to the preamble of the independent claim.

### BACKGROUND OF THE INVENTION

The human vascular system may suffer from a number of problems. These may broadly be characterised as cardiovascular and peripheral vascular disease. Among the types of disease, atherosclerosis is a particular problem. Atherosclerotic plaque can develop in a patient's cardiovascular system. The plaque can be quite extensive and occlude a substantial length of the vessel.

A technique used to identify and measure the extent of the stricture caused by plaque is to measure the pressure inside the vessel in the part of the vessel where the stricture is located. In the prior art there are numerous examples of catheters suitable to perform pressure measurements. Among those may be mentioned U.S. Pat. No. 6,615,667 related to a guidewire provided with a combined flow, pressure and temperature sensor.

In order to further improve the methods used to identify strictures of vessels the inventor has identified a technique applicable for presenting obtained measured results in a more user-friendly way.

Thus, the object of the present invention is to achieve a pressure measurement system provided with means arranged to present obtained pressure data in a more user-friendly way.

### SUMMARY OF THE INVENTION

The above-mentioned object is achieved by the present invention according to the characterizing portion of the independent claim.

Preferred embodiments are set forth in the dependent claims.

The vascular pressure sensor guidewire of the present invention, is adapted for the identification, measurement and diagnosis of vascular tissue, in particular, of atherosclerotic plaque. Treatment may be effected by reinserting a prior art catheter apparatus to a predetermined area of the vascular tissue. This reinsertion may be achieved in a controlled manner as the prior pressure scan with the device used to produce a pressure map of the vascular tissue. This information may be stored in an external processing means and can be used to relocate the area of risk. This procedure requires less contrast media to be infused into the patient than would normally be required in similar vascular interventional procedures as the position of the vascular catheter is known due to the data stored in the processing means. The pull-back device may then, under the control of a user, be used to drive the catheter back to, for example, the starting point of the pressure measurement or any point along the path of the pressure data acquisition, for treatments of the vascular tissue.

For example, the catheter apparatus used to treat the area may be by any of the usual therapeutic procedures, including localised delivery of a therapeutic agent, delivery of a stent, brachy therapy, ablation of selected tissue etc. Furthermore, the pressure sensor guidewire may additionally comprise angioplasty balloons or sleeves.

According to a preferred embodiment of the present invention, pressure data is obtained in a vascular vessel by withdrawing a pressure sensor guidewire that senses the pressure in the vessel over a predetermined length of the vessel and processing pressure data with reference to image data representative of the vascular morphology to provide an integrated graphical image output in which the pressure data is mapped onto a corresponding position on the image where that pressure data was detected.

Preferably, the image data is angiogram image data and the pressure data is captured using a vascular pressure sensor guidewire. This combined display of angiogram image data and pressure data is considered advantageous in that the physician immediately and more accurately may identify areas of risk in the vessel.

Alternatively, the image data may be obtained by using intravascular ultrasound (IVUS) technique.

The invention is naturally also applicable in combination with any imaging technique capable of generating a two-dimensional image of the blood vessel.

The integrated graphics image output is a two-dimensional representation of a target vessel with a pressure profile of the target vessel overlaid.

Furthermore, in particular if the IVUS technique is used, the integrated graphics image output may also be a three-dimensional representation of the target vessel with a pressure profile of the target vessel overlaid.

Thus, the present invention relates to a pressure measurement system, comprising a pressure detection device comprising a guidewire provided with a pressure sensor connected to an electrical carrier for transmitting pressure data from the sensor to a remote device. The guidewire is adapted to be inserted into a vessel of a subject. The system further comprises an image data capturing device for capturing image data representative of the vessel. The system in addition comprising a computer program product which comprises computer executable instructions for manipulating image data and pressure data to generate an output in which the pressure data is mapped onto a corresponding position on an image in order to provide an integrated graphical image output, wherein the pressure data is data from the pressure detection device and represents the pressure inside the vessel and the image data is representative of the vessel.

The pressure data may be displayed according to a number of different alternatives.

Preferably, a numeric relation is calculated between a static reference pressure, obtained proximally the stenosis, and the pressure data, and the numeric relation is displayed at the corresponding position of the image where large and fast pressure changes are indicated, e.g. according a colour scale in combination with the pressure value.

A graphical pressure profile may be displayed along the vessel where the stenosis may be identified.

Fractional Flow Reserve (FFR) values may also be calculated and displayed along the vessel, see e.g. U.S. Pat. No. 6,565,514.

A major advantage of the present invention is that this will make it possible to indicate the significance of the stenosis.

### SHORT DESCRIPTION OF THE APPENDED DRAWINGS

FIG. 1 is a schematic diagram of a system for conducting vascular catheterisation of a patient.

FIG. 2 is a schematic flow diagram of the important steps performed by the pressure measurement system according to the present invention.

FIG. 3 shows a vessel of a subject where plaque is identified.

FIG. 4 shows the vessel of FIG. 3 as it is displayed in accordance with the present invention.

### DETAILED DESCRIPTION OF PREFERRED EMBODIMENTS OF THE INVENTION

Whilst in normal circumstances the pressure sensor guidewire provided with a pressure sensor is inserted manually, it is intended that when performing vascular measurements the pressure sensor guidewire is pulled back relative to a predetermined start position, preferably by using an electromechanical pull-back drive coupled to the body of the catheter. EP-1291034 discloses a typical pull-back mechanism that may be used in connection with the pressure sensor guidewire when implementing the present invention.

The pressure sensor guidewire is inserted such that the start position is reached when pressure sensor is positioned distally the stenosis to be measured.

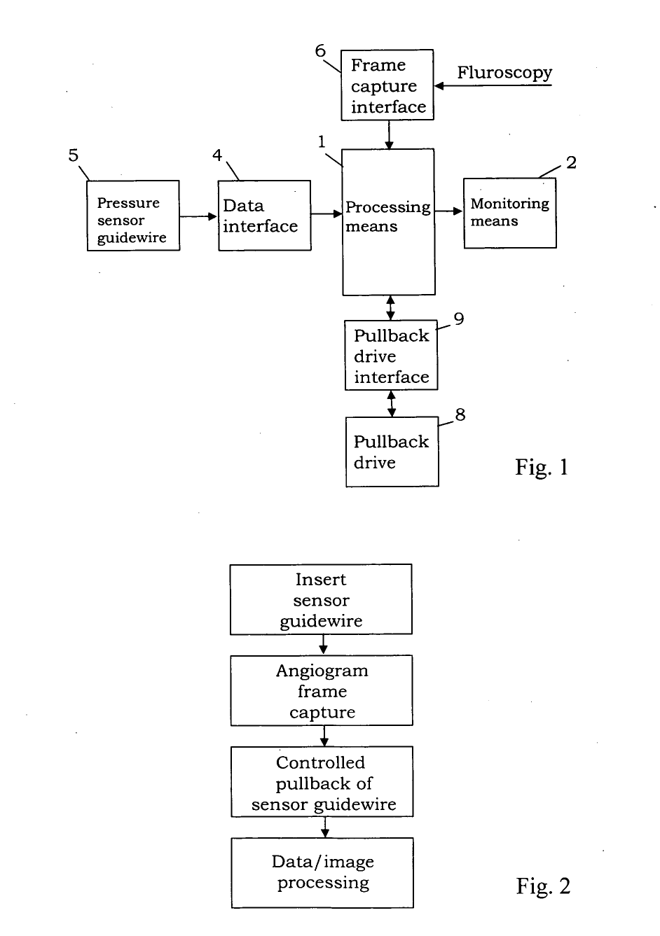

The pull-back drive 8 (FIG. 1) may be controlled by a processing means 1 via a pull-back drive interface 9. The system software accesses user-defined configuration files to get the necessary information about controlling the pull-back interface. Data sampling rate, recording duration and pre-selected retraction rate are taken into consideration for adjusting the pull-back speed. The speed may naturally be varied in dependence of the specific situation but as a general rule the speed is adjusted such that the pull-back procedure lasts for approximately 10-20 seconds.

The pressure is continuously measured, preferably at a sampling rate of 100 Hz, during the pull back procedure. The software routines control a D/A converter that feeds the input of the pull-back interface with an appropriate control voltage.

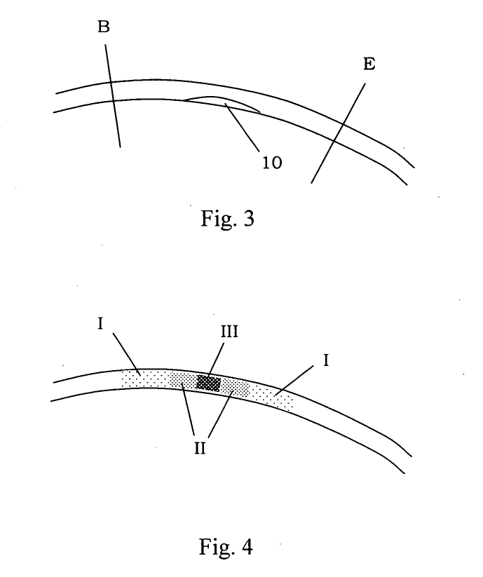

In general a measurement session includes obtaining a static reference pressure proximally the stenosis and then relating the pressure data obtained when drawing the pressure sensor from a position distally the stenosis, to that static reference pressure. The part of the vessel chosen to be further investigated is identified in FIG. 3 to be between B (beginning) and E (end).

Pressure data plotting may be both on-line and/or off-line. In an on-line mode, the monitoring means 2 presents a pressure/time-distance graph, where pressure is continuously plotted as connected dots. In an off-line mode, pressure data can be loaded from the data store and plotted on the screen graph. The user can scroll to different time/pressure locations, while several automated functions may be provided, for example automatic min-max marking, colour coding of the pressure.

With references to FIG. 1 the system includes a processing means 1, e.g. a personal computer (PC) that presents a graphical user interface (GUI) via one or many monitoring means 2. Preferably, the user interface system is based on a Microsoft Windows™ platform. Multiple windows may be used to acquire/project data from/to the user. Although not shown, the PC can accept user inputs via a keyboard and mouse, or other pointing device, in the usual manner. The PC may include a number of data stores, which may be external, and a CD ROM reader/writer device.

The PC is coupled via a data interface 4 to guidewire 5 of the pressure detection device, details of which will be described below. In this example, the guidewire 5 transmits pressure data from a pressure sensor which are received by the data interface 4. An analogue pressure data signal is converted to a digital signal using an A/D converter within the data interface 4 at a user configured sampling rate of up to 2,5 KHz. Typically, as mentioned above, the sampling rate would be set at around 100 Hz to reduce the quantity of data acquired.

The data interface 4 receives the pressure data and couples it to the PC over a PCI bus. The pressure data is written into an area of memory within the data store reserved for pressure data where they can subsequently be retrieved for data processing along with the corresponding time sequenced data from other channels and image data from other sources.

Preferably, the pressure data from the pressure sensor guidewire 5 are introduced to the system software running on the PC using function calls. Pressure data are input to the software as the actual voltage at the A/D hardware inputs, and therefore they have to be converted to pressure. A sensor data convert function handles this process.

According to a preferred embodiment the system is designed to be used in conjunction with a fluoroscopy x-ray apparatus and therefore includes a video frame capture interface 6 that couples fluoroscopy video data inputs to the PC via a PCI bus. Similarly, it can be used in conjunction with intravascular ultra-sound (IVUS) image data fed from the pressure sensor guidewire 5 (when provided with the appropriate hardware). The system software allocates sufficient memory area to the system's memory for this data, taking into account the current system configuration, for example sampling rate, recording time, and video frame size.

In a preferred embodiment memory handle hDib is used to map video data directly through the PCI bus from the video frame capture interface 6 to this allocated area in memory. hDib memory is divided into i equal chunks, each of a size equal to the frame capture interface frame-puffer. Optionally, hDib [i] data can also be mapped to a memory area of a screen-video buffer, giving capability of live preview during recording. Each time the software records an x group of pressure measurements, it prompts for a frame capture at hDib [x]. A user configuration file determines the ratio between pressure data fluoroscopy video frame capture.

Whilst in normal circumstances the pressure sensor guidewire 5 is inserted manually, it is intended that when performing vascular measurements the guidewire 5 is pulled back relative to a predetermined start position using an electromechanical pull-back drive 8 coupled to the body of the guidewire. The pull-back drive 8 is controlled by the PC via a pull-back drive interface 9. The system software accesses user-defined configuration files to get the necessary information about controlling the systems automatic pull-back interface 9. Data sampling rate, recording duration and pre-selected retraction rate are taken into consideration for adjusting the pull-back speed. The software routines control a D/A converter (not shown) that feeds the input of the pull-back interface 9 with an appropriate control voltage. The controlled pull-back process will be described in more detail below.

Pressure data plotting may be both on-line and/or off-line. In an on-line mode, the monitor presents a pressure/time-distance graph, where pressure is continuously plotted as connected dots. In an off-line mode, pressure data can be loaded from the data store (or other media) and plotted on the screen graph. The user can scroll to different time/pressure locations, while several automated functions may be provided, for example auto min-max marking, colour coding of pressure with colours varying from e.g. dark-blue (minimum pressure) to flashing-red (maximum pressure). A separate window may show numeric details for the particular time/distance position. Video frame data from simultaneous fluoroscopy/IVUS may be plotted as image frames in a separate window. By moving to a specific time/pressure position, the corresponding video frame is automatically projected. In this way, pressure and video frames are accurately synchronised.

The system software is designed to provide basic and advanced image processing functions for the captured fluoroscopy/IVUS video frames, such as filtering and on-screen measurement functions. The user can filter the captured frame to discard unwanted information while focusing on the desired one. There are several auto-filter options as well as manual adjustment of the image curve. In addition, the user can calibrate the system and proceed in performing on-screen measurements of both distances and/or areas. Automatic routines perform quantification of the measurements giving significant information on lesion characteristics. The pressure can also be colour coded on the fluoroscopy frame, providing unique information about the correlation between pressure and morphology.

With reference to FIG. 2, in use, the sequence of events begins with the insertion of a guiding catheter into the area of general interest, for example the cardiac region. Where, for example, the coronary arteries are to be examined, the guiding catheter is inserted so that it is in or adjacent to the opening of the coronary arteries. The pressure sensor guidewire is then inserted into the coronary artery, past the point of specific interest. The guidewire is usually inserted with the aid of standard fluroscopic techniques, as is the guiding catheter. The pressure sensor guidewire of the present invention is then manoeuvred to a position beyond the specific area of interest in the coronary artery with the aid of fluoroscopy.

An angiogram is taken to assess the position of the guidewire in the vascular tissue. This image is saved and the position of the guidewire is marked on the image so as to define a starting point for the controlled pull-back step.

The guiding catheter is then locked in position and both the sheath and the lumen housed in the sheath are locked to mounts on the pull-back device. Controlled pull-back of the pressure sensor guidewire then takes place. The pull-back takes place at a constant speed and is controllable by the user. Pull-back typically takes place at speeds of 0.1 to 2 mm in divisions of 0.1 mm or so.

The pull-back takes place over a distance of the vascular tissue being measured. Pressure readings may be taken intermittently or substantially continuously. The data transmitted by the sensor is captured for data and image processing together with a fluoroscopy/IVUS image frame.

As mentioned above, the system software has the capability to capture image-frames that come from standard fluoroscopy or IVUS devices simultaneously with pressure. Spatial data that come from fluoroscopy/IVUS are combined by the software with pressure data. This is done as follows: Before the pressure measurement procedure starts, and while the guidewire is still out of the target vessel, the user records the fluoroscopy-tube/bed position and records a video frame during injection of contrast media. The vessel is opacified, and the image is stored and projected on one of the system monitors. The user calibrates the pixel/mm relation by using the guiding catheter as a known reference so that distances in mm can subsequently be estimated accurately on the monitor.

As shown in FIG. 3 showing a vessel of a subject where plaque 10 is identified, the user then marks the beginning and ending of the area of interest (points B and E) by clicking on them using the mouse: in return, the software marks these points on the monitor by arrows or lines. The user then positions the pressure sensor guidewire in the target vessel by pushing it forward in the guiding catheter until the fluoroscopic marker on the pressure sensor guidewire passes point E over a few mm; while watching the system's monitor, the user manually pulls the guidewire back gently until the fluoroscopic marker overlaps exactly on point E. The software then instructs the automatic pull-back device to pull back the pressure sensor guidewire over the length of the BE curve within the vessel.

The software then performs auto-border detection on the BE area of the fluoroscopy video frame using e.g. a photoluminescence technique, and pressure is subsequently coded in the atherosclerotic plaque outline as RGB colour degradation from dark-blue (0,0,255) corresponding to the minimum detected pressure, to flashing red (255,0,0) corresponding to the maximum detected pressure. A reference colour map may be provided, and by moving the mouse cursor inside the BE area, pressure values may also automatically be provided in a numeric format.

FIG. 4 shows the same vessel as in FIG. 3 where the different pressure values are indicated by different degrees of shading. The darkest shading (III) represents the highest pressure and the brightest shading (I) represents the lowest pressure.

The present invention is not limited to the above-described preferred embodiments. Various alternatives, modifications and equivalents may be used. Therefore, the above embodiments should not be taken as limiting the scope of the invention, which is defined by the appending claims.

1. Pressure measurement system, comprising a pressure detection device comprising a pressure sensor guidewire (5) provided with a pressure sensor connected to an electrical carrier for transmitting pressure data from the sensor to a processing means (1), said guidewire is adapted to be inserted into a vessel of a subject, and an image data capturing device for capturing image data representative of the vessel,
characterized in that the processing means (1) comprising a computer program product which comprises computer executable instructions for manipulating image data and pressure data to generate an output in which the pressure data is mapped onto a corresponding position on an image where that pressure data was detected to provide an integrated graphical image output on a monitoring means (2), wherein the pressure data is data from the pressure detection device and represents the pressure inside the vessel and the image data is representative of the vessel.

2. Pressure measurement system according to claim 1, wherein the pressure detection device is drawn continuously along the section of the vessel under examination as pressure data are recorded.

3. Pressure measurement system according to claim 2, wherein the pressure detection device is drawn by an external pull-back device.

4. Pressure measurement system according to claim 3, wherein the speed is adjusted such that the pull-back procedure lasts for approximately 10-20 seconds.

5. Pressure measurement system according to claim 1, in which individual pressure measurements are taken along a predetermined length of the vessel and related to a reference pressure value sensed proximally the measurement site of the vessel.

6. Pressure measurement system according to claim 1, wherein the image data capturing device uses fluoroscopy.

7. Pressure measurement system according to claim 1, wherein the image data capturing device uses intravascular ultrasound (IVUS).

8. Pressure measurement system according to claim 1, wherein the pressure data is presented by colour coding the pressure data.

9. Pressure measurement system according to claim 1, wherein Fractional Flow Reserve (FFR) values are calculated and displayed along the vessel.
